(19) United States
(12) Patent Application Publication
(10) Pub. No.: US 2009/0149943 A1
(43) Pub. Date: Jun. 11, 2009

(54) FLEXIBLE STENT

(75) Inventor: Allen Tower, North Lawrence, NY (US)

Correspondence Address: Hiscock & Barclay, LLP, One Park Place, 300 South State Street, Syracuse, NY, 13202-2078, US

(73) Assignee: NUMED, INC., Nicholville, NY (US)

(21) Appl. No.: 11/951,595

(22) Filed: Dec. 6, 2007

(51) Int. Cl. A61F 2/86

(52) U.S. Cl. 623/1.16

(57) ABSTRACT

This invention involves a radially expandable stent that has wire wound circular sections that are cojoined to create a cylinder by a series of single strand wire hinges. The hinges are situated to provide the stent created by the expanded sections with enhanced flexibility without sacrificing the overall support strength of the stent.

### FIELD OF THE INVENTION

This invention relates to a percutaneously implantable stent having improved flexibility that allows the stent to flex in a number of directions without the loss of the strength.

### BACKGROUND OF THE INVENTION

Intervascular stents that are capable of being percutaneously implanted via a blood vessel are typically carried into the treatment site upon a balloon catheter with the stent in a collapsed condition upon the balloon. Once the stent is properly located within the site, the balloon is inflated to expand the stent into an operable position. Once brought into the operative position the stent locks in place and little more can be done to change the rigid expanded posture. This renders the stent less than suitable for use in association with a child who over time can grow to a size such that the stent is no longer reliable.

It should be further noted, that the circular sections of wire wound stents found in the prior art are generally held together by welds. Accordingly as the stent is expanded from a collapsed position into an expanded position, the axial length becomes foreshortened. This, in turn, makes positioning of the stent within a body cavity somewhat difficult.

Lastly, wire wound stents, when expanded, typically form relatively rigid bodies with the two ends of the expanded cylinder presenting rather sharp corners to the surrounding body tissue. Over time these corners can become embedded in the surrounding tissue causing further problems. Because of its rigidity, a wire wound stent has a greater tendency to become embedded in surrounding tissue when it is implanted in body cavities having less than smooth linear surfaces.

### SUMMARY OF THE INVENTION

It is therefore a primary object of the present invention to improve intravascular stents.

It is a further object to provide greater flexibility to an intravascular stent without sacrificing the stents overall strength.

Another object of the present invention is to provide a flexible stent that does not become foreshortened as it is expanded.

Yet another object of the present invention is to provide a stent that can be further expanded beyond its initial implantation posture.

A still further object of the present invention to provide a stent having the ability to more closely follow the contours of a body cavity in which it is implanted.

Yet a further object of the present invention is to provide a stent that can be collapsed into a very compact package to better facilitate the percutaneous implantation of the stent.

These and other objects of the present invention are attained by an expandable and collapsible stent that includes a series of wire wound circular sections that are coaxially aligned along a common axis to establish a cylinder. Each section is formed of a strand of sinusoidally shaped wire containing a series of circumferentially spaced bends, the tips of which define the sides of each section. Each of the bend tips are positioned adjacent to a bend tip in a neighboring section and selected adjacent tip pairs are co-joined by a wire hinge made up of a single strand of wire that is capable of flexing in a number of different directions.

### BRIEF DESCRIPTION OF THE DRAWINGS

For a better understanding of these and other objects of the present invention, reference should be made to the following detailed description of the invention which is to be read in association with the accompanying drawings, wherein:

FIG. 1 is a side elevation illustrating a stent embodying the present invention mounted upon a balloon catheter delivery system;

FIGS. 2-5 illustrate various single wire hinge configurations that can be employed in a stent embodying the present invention;

FIGS. 6-8 are zig maps illustrating different wire wound stent patterns embodying the present invention;

FIG. 9 is a partial side elevation in section showing a prior art stent implanted within a curved section of a body cavity; and

FIG. 10 is an enlarged side elevation in section showing a stent embodying the present invention implanted in the curved section of a body cavity.

### DETAILED DESCRIPTION

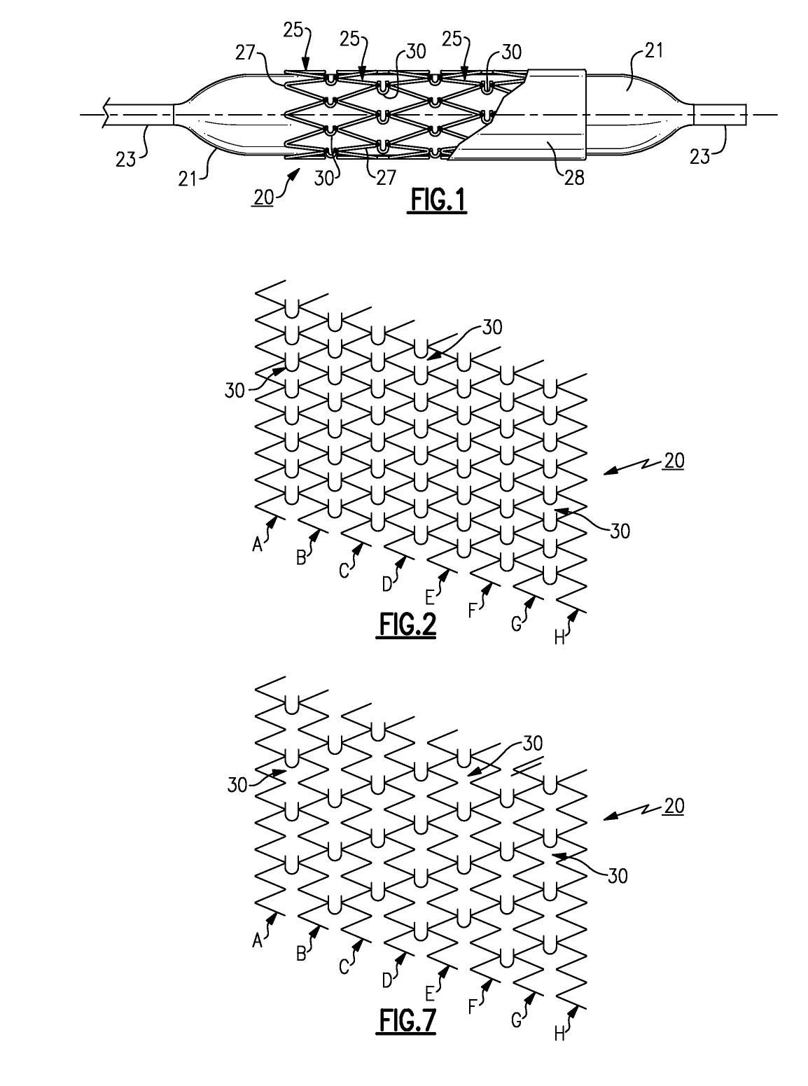

The present stent is shown in FIG. 1 in an inflated condition. The stent includes a number of circular wire wound sections 25-25 that are coaxially aligned along a center axis 26 to establish a cylinder of given length. Each section, in turn, contains a single strand of wire that is in the form of a sinusoid that passes back and forth along the circumference of the section. Each wire sinusoid has a series of uniformly spaced bends, the tips 27 of which describe the opposed side boundaries of the sections. In assembly, the sections are aligned so that tip running along one side of a section are located adjacent to and in close proximity with tips contained in a neighboring section. Selected ones of the adjacent tip pairs are connected together by a single wire hinge 30. Preferably at least two hinges are used to connect each of the sections to its neighbor to hold the sections in a cylindrical alignment. The stent may be covered with a bio-compatible sleeve 28 fabricated of Teflon or the like.

In this embodiment of the invention the adjacent tips of the selected bends are cojoined by U-shaped hinges 30 one of which is shown in further detail in FIG. 3. Each U-shaped hinge member 30 is positioned in the space separating two adjacent tips 27-27. One leg 31 of the hinge is welded to one of the adjacent tips and the opposite leg 32 is welded to the other adjacent tip. Preferably the hinge is fabricated of the same material as the wire wound sections which may be but not limited to an annealed alloy made of 90% platinum and 10% iridium. Any other material that is known and used in the construction of wire wound stents may also be utilized without departing from the teachings of the present invention. The hinges, however, can be fabricated of material having a shear stress that is about equal to that of strand making up the sections so that the hinge can be bent without breaking under normal load conditions.

Turning to FIG. 2, there is illustrated a zig map showing a flat layout of the stent 20 illustrated in FIG. 1. In this particular case, the stent contains eight circular sections which appear on the map as flat parallel columns identified as columns A-H. Each column or section contains eight bend tips running along each side of the column. At closure, the two terminal ends of each column are brought together to form a single bend. As the columns move from left to right on the map, the terminal tips each dropped down a space equal to the space separating the tips. Accordingly, the closure points in each of the sections do not become aligned along the length of the cylinder. As noted above, in this embodiment there are eight tips equally spaced along the opposed sides of each section and each of the adjacent pair of tips is cojoined by a single strand wire hinge 30 in the manner set forth above.

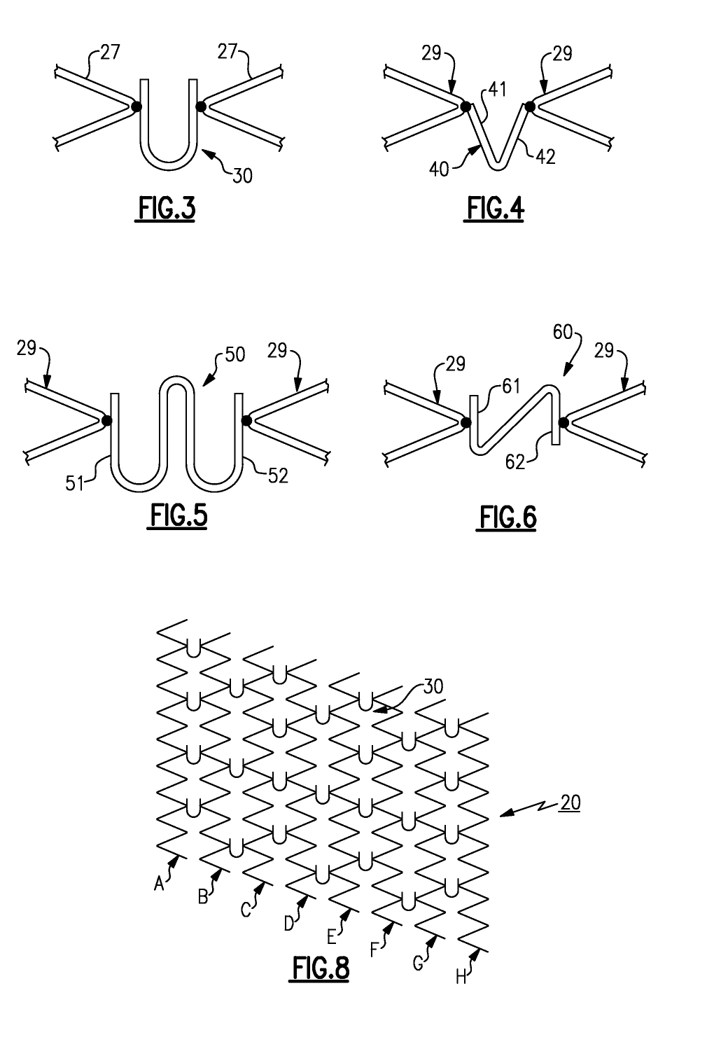

The flexibility of the stent may be further selectively altered by changing the configuration and/or the diameter of the hinges. As illustrated in FIG. 4, a hinge 40 may be V-shaped having a pair of legs 41 and 42 that are welded to the tips 29-29 of adjacent bends. FIG. 5 illustrates a W-shaped hinge 50 wherein the outside legs 51 and 52 are welded to a pair of adjacent tips 29-29. A Z-shaped hinge 60 is illustrated in FIG. 6 in which the upper leg 61 and lower the leg 62 of the hinge are again welded to a pair of adjacent tips 29-29.

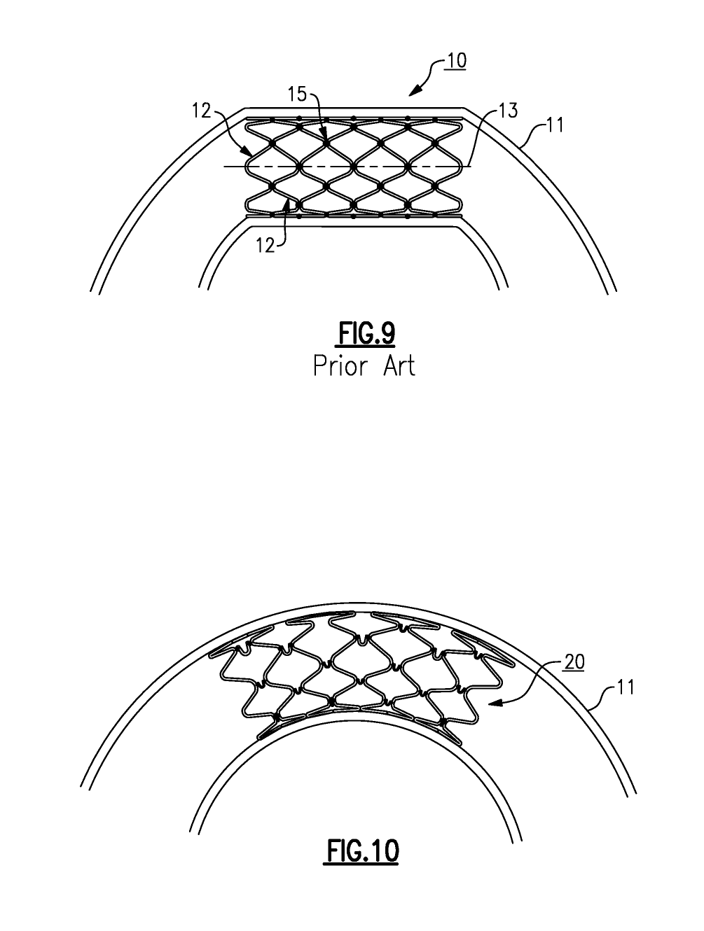

FIG. 7 is also a zig map that illustrates a second embodiment of the invention. Here again, the structure contains eight columns A-H each of which contains eight equally spaced bend tips running along each side of the columns. In this embodiment every other adjacent tip pair is cojoined by a hinge 30 and in each column the four hinges are offset from its neighbor by a space equal to the spacing between tips. Accordingly, every other adjacent tip pair is not connected and is allowed to float thus providing the stent with greater flexibility than that described above with reference to FIG. 2.

FIG. 8 is a further zig map illustrating another embodiment of the invention. In this embodiment, there are again eight columns A-H each of which describes a wire wound section of the stent 20 with each column containing eight equally spaced tips extending along each side of the column. Unlike the first two embodiments shown in FIGS. 6-7, an unequal number of hinges 30 are employed to cojoin the adjacent tip pairs in the neighboring columns. For example, four equally spaced hinges are used to connect columns A and B while only three hinges are used to connect columns B and C. This pattern is repeated for columns C and D and D and E and so on as the columns move from left to right on the map. Accordingly, the stent that is established at closure will have less hinges than the first two stents and will thus have greater flexible than that illustrated in FIG. 7.

As can be seen any number of hinge placement patterns may be selected to construct stents having differing rigidities or to provide sections having more or less flexibility along the length of the stent that might best satisfy a particular application.

FIG. 10 shows a stent 20 that embodies the teachings of the present invention implanted within a curved section of a blood vessel. As can be seen, the individual sections making up the stent are able to position themselves independently with regard to the bend contour so that the shape of the stent conforms closely to that of the bend thereby minimizing the chances of the stent from becoming deeply embedded in the surrounding tissue.

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

1. An expandable and collapsible stent that includes:
a series of circular sections that are coaxially aligned along a common center line to create a cylinder;
each of said section being formed of a single strand of sinusoidally shaped wire having a series of circumferentially spaced bends having tips that define the opposed sides of each of said sections;
said tips on one section being positioned in close proximity with tips on a neighboring section to establish an adjacent tip pair; and
a single strand wire hinge cojoining at least two, adjacent tip pairs between each of said neighboring sections.

2. The expandable or collapsible stent of claim 1 wherein said wire hinge is a U-shaped member having one leg attached to one tip of said at least one adjacent tip pair and an opposing leg attached to a second tip of said at least one adjacent tip pair.

3. The expandable and collapsible stent of claim 1, wherein said wire hinge is a V-shaped strand having one leg secured to one tip of said at least one adjacent tip pair and a second leg secured to the other tip of said at least two adjacent tip pair.

4. The expandable and collapsible stent of claim 1, wherein said wire hinge is a W-shaped strand having one outer leg secured to one tip of said at least one adjacent tip pair and the second outer leg secured to the other tip in said at least two adjacent tip pair.

5. The expandable and collapsible stent of claim 1, wherein said wire hinge is a Z-shaped member having an upper leg secured to one tip of said at least one adjacent tip pair and a lower leg secured to the other tip in said at least two adjacent tip pair.

6. The expandable and collapsible stent of claim 1, wherein each of the adjacent tip pairs between neighboring sections is co-joined by a single strand wire hinge.

7. The expandable and collapsible stent of claim 1, wherein one half of said adjacent tip pairs between neighboring sections are co-joined by a single strand wire hinge.

8. The expandable and collapsible stent of claim 1, wherein said tips of each section are uniformly spaced over the circumference of each section and the adjacent tips on neighboring sections are longitudinally aligned along the length of said cylinder.

9. The expandable and collapsible stent of claim 8, wherein wire hinges co-join every other adjacent pair of tips about the circumference of each section.

10. The expandable and collapsible stent of claim 1, having a plurality of wire hinges co-joining each neighboring section and wherein the number of wire hinges varies between each of the neighboring sections.

11. The expandable and collapsible stent of claim 10, wherein the number of wire hinges vary by one between successive neighboring sections.

12. The expandable and collapsible stent of claim 1, wherein said at least one single strand wire hinge is fabricated of the same material as said sinusoidal wire strands of said sections.
